(19) United States
(12) Patent Application Publication
(10) Pub. No.: US 2017/0020463 A1
(43) Pub. Date: Jan. 26, 2017

(54) PHYSIOLOGY MEASURING SYSTEM AND METHOD THEREOF

(71) Applicant: INDUSTRIAL TECHNOLOGY RESEARCH INSTITUTE, HSINCHU (TW)

(72) Inventors: HONG DUN LIN, TAIPEI CITY (TW); YEN HSIEN LEE, TAOYUAN COUNTY (TW)

(21) Appl. No.: 15/285,241

(22) Filed: Oct. 4, 2016

Related U.S. Application Data
(62) Division of application No. 13/713,768, filed on Dec. 13, 2012.

(51) Int. Cl. A61B 5/00; A61B 5/021

(52) U.S. Cl. CPC A61B 5/7278; A61B 5/02116; A61B 5/0004; A61B 5/02125; A61B 5/742

(57) ABSTRACT

A method processed by a signal processing device of a physiology measuring system includes calculating a pulse time difference of a first pulse peak and a second pulse peak; calculating a pulse wave velocity; and calculating a systolic blood pressure of an artery, and a diastolic blood pressure of the artery; wherein the first pulse peak and the second pulse peak is generated by a sensing device; wherein the sensing device emits a plurality of first radiated pulse signals to a first measure point of the artery and, in turn, receiving a plurality of first scattered pulse signals reflected from the first measure point of the artery; and wherein the sensing device emits a plurality of second radiated pulse signals to a second measure point of the artery and, in turn, receiving a plurality of second scattered pulse signals from the second measure point of the artery.

### CROSS-REFERENCE TO RELATED APPLICATIONS

This application is a divisional of U.S. application Ser. No. 13/713,768, filed Dec.13, 2012.

### TECHNICAL FIELD

The current disclosure relates to physiology measurement and, in particular, to a physiology measuring system and method thereof.

### BACKGROUND

In current blood pressure measuring devices, auscultation and electron resonance, with a cuff, are widely applied to measure the systolic and diastolic blood pressure of an artery.

Therefore, the cuff needs to be inflated and deflated for indirectly measuring non-continuous blood pressure. However, when measuring continuous blood pressure, the cuff needs to be setup correctly and be inflated and deflated repetitively, which would cause a great inconvenience to the users, and as such, the feasibility and practicality would be significantly less effective.

The current disclosure discloses a physiology measuring system and method thereof.

### SUMMARY

In accordance with one embodiment of the current disclosure, a method processed by a signal processing device for a physiology measuring system, includes calculating a pulse time difference of a first pulse peak and a second pulse peak; calculating a pulse wave velocity; and calculating a systolic blood pressure of an artery, and a diastolic blood pressure of the artery, wherein the first pulse peak and the second pulse peak is generated by a sensing device; the sensing device emits a plurality of first radiated pulse signals to a first measure point of the artery and, in turn, receiving a plurality of first scattered pulse signals reflected from the first measure point of the artery; the sensing device emits a plurality of second radiated pulse signals to a second measure point of the artery and, in turn, receiving a plurality of second scattered pulse signals from the second measure point of the artery. In order to provide further understanding of the techniques, means, and effects of the current disclosure, the following detailed description and drawings are hereby presented, such that the purposes, features and aspects of the current disclosure may be thoroughly and concretely appreciated; however, the drawings are provided solely for reference and illustration, without any intention to be used for limiting the current disclosure.

### BRIEF DESCRIPTION OF THE DRAWINGS

A more complete understanding of the current disclosure may be derived by referring to the detailed description and claims when considered in connection with the Figures, where like reference numbers refer to similar elements throughout the Figures, and:

FIG. 1 is a schematic view of a sensing device 10 of one embodiment of the current disclosure;

FIG. 2 is a schematic view of a physiology measuring system 20 of one embodiment of the current disclosure;

FIG. 3 shows a schematic view of a detailed circuit of a physiology measuring system of one embodiment of the current disclosure;

FIG. 4 shows a schematic view of a detailed circuit of a physiology measuring system of one embodiment of the current disclosure;

FIG. 5 shows a schematic view of time differences of a radiated pulse signal and a scattered pulse signal;

FIG. 6 shows a schematic wave form of first pulse peaks and second pulse peaks of one embodiment of the current disclosure; and

FIG. 7 shows a flow chart of a method of a physiology measuring system of one embodiment of the current disclosure.

### DETAILED DESCRIPTION

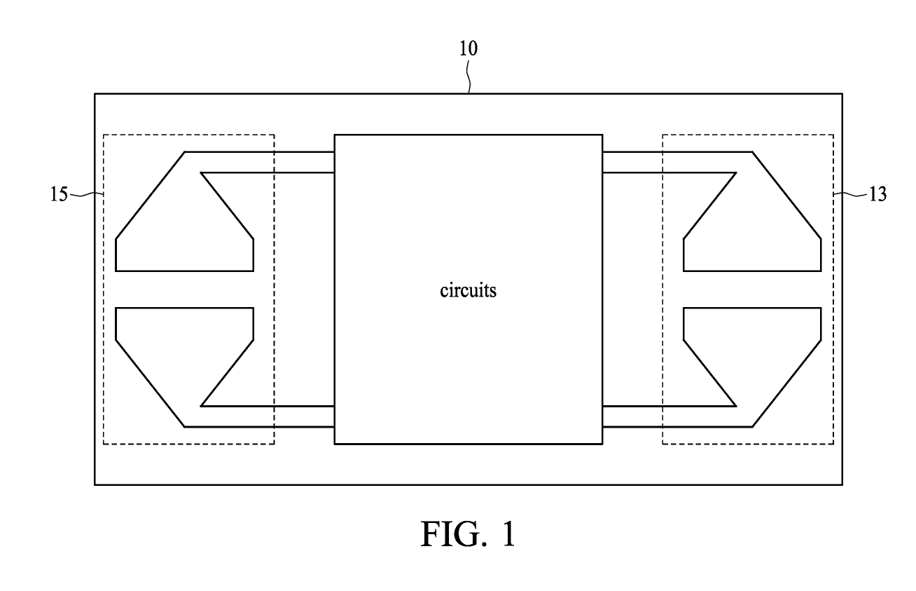

FIG. 1 is a schematic view of a sensing device 10 of one embodiment of the current disclosure. As shown in FIG.1, the sensing device 10 comprises a first antenna 13 and a second antenna 15, wherein the first antenna 13 is configured to be disposed on an end of the sensing device 10 and the second antenna is configured to be disposed on an opposite end of the sensing device 10. In other embodiments, the first antenna 13 and the second antenna 15 are not limited to those disposed on opposite ends.

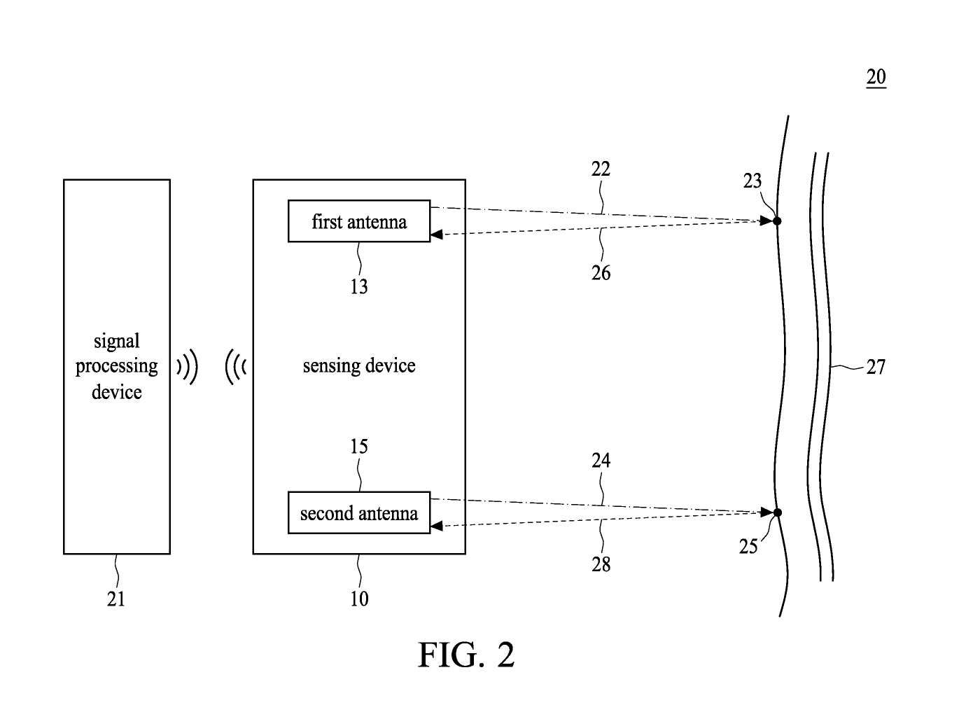

FIG. 2 is a schematic view of a physiology measuring system 20 of one embodiment of the current disclosure. As shown in FIG. 2, the physiology measuring system 20 comprises a signal processing device 21, and the sensing device 10.

FIG. 2 illustrates a contactless embodiment. In other embodiments, however, the application is not limited to such contactless implementation.

The signal processing device 21 communicates with the sensing device 10 via a wireless protocol, which may include a Bluetooth protocol. Moreover, referring to FIG. 2, the first antenna 13 is configured to emit a plurality of first radiated pulse signals 22 and, in turn, receive a plurality of first scattered pulse signals 26. Each of the first scattered pulse signals 26 is a reflection signal, after each of the first radiated pulse signals 22 hits a first measure point 23 of an artery. According to one embodiment, the signal processing device 21 comprises a desktop or a portable electronic device.

The second antenna 15 is configured to emit a plurality of second radiated pulse signals 24 and, in turn, receive a plurality of second scattered pulse signals 28, wherein each of the second scattered pulse signals 28 is a reflection signal, after each of the second radiated pulse signals 24 hits a second measure point 25 of the artery. The first measure point 23 and the second measure point 25 are away from each other at a distance D. According to one embodiment, the radiated pulse signals are 5 ns.

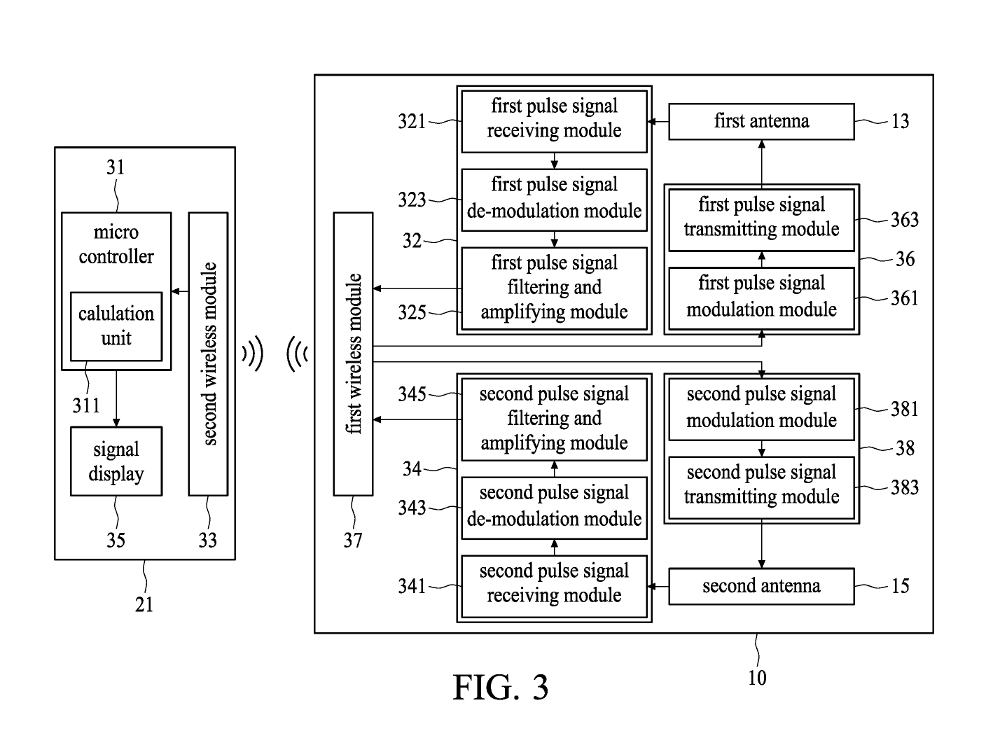

FIG. 3 shows a schematic view of a detailed circuit of a physiology measuring system of one embodiment of the current disclosure. As shown in FIG. 3, the signal processing device 21 further comprises a second wireless module 33, a microcontroller 31 and a signal display 35.

The microcontroller 31 comprises a calculation unit 311. The sensing device 10 further includes the first antenna 13, the second antenna 15, a first pulse signal receiver 32, a second pulse signal receiver 34, a first pulse signal generator 36, a second pulse signal generator 38, and a first wireless module 37.

The first pulse signal receiver 32 may include a first pulse signal receiving module 321, a first pulse signal de-modulation module 323, and a first pulse signal filtering and amplifying module 325. The second pulse signal receiver 34 may include a second pulse signal receiving module 341, a second pulse signal de-modulation module 343, and a second pulse signal filtering and amplifying module 345.

The first pulse signal generator 36 may include a first pulse signal modulation module 361 and a first pulse signal transmitting module 363. The second pulse signal generator 38 may include a second pulse signal modulation module 381 and a second pulse signal transmitting module 383.

Referring back to FIG. 2, the first pulse signal generator 36 is configured to generate the first radiated pulse signals 22, according to a generating instruction of a first radiated pulse signal from the signal processing device 21 via the first wireless module 37, to the first antenna 13. The second pulse signal generator 38 is configured to generate the second radiated pulse signals 24, according to a generating instruction of a second pulse signal from the signal processing device 21 via the first wireless module 37, to the second antenna 15.

The first radiated pulse signals 22 may be modulated by the first pulse signal modulation module 361, and then, be sent to the first pulse signal transmitting module 363. The second radiated pulse signals 24 may be modulated by the second modulation module 381, and then, be sent to the second transmitting module 383.

The first scattered pulse signals 26, from the first pulse signal receiving module 321, may be de-modulated by the first de-modulation module 323, and be filtered and amplified by the first pulse signal filtering and amplifying module 325, before being sent to the signal processing device 21 via the first wireless module 37.

Furthermore, the second scattered pulse signals 28, from the second pulse signal receiving module 341, may be de-modulated by the second de-modulation module 343, and be filtered and amplified by the second pulse signal filtering and amplifying module 345, before being sent to the signal processing device 21 via the first wireless module 37.

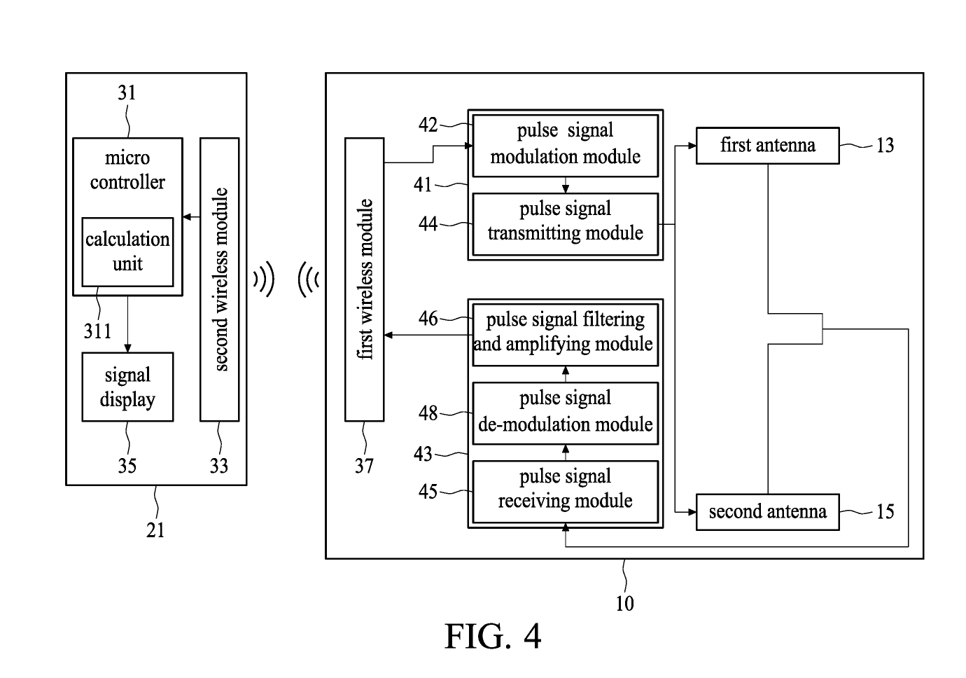

FIG. 4 shows a schematic view of a detailed circuit of a physiology measuring system of one embodiment of the current disclosure. Compared with FIG. 3, the physiology measuring system includes a pulse signal generator 41 and a pulse signal receiver 43.

As shown in FIG. 4, the pulse signal generator 41 further comprises a pulse signal modulation module 42 and a pulse signal transmitting module 44. The pulse signal receiver 43 further comprises a pulse signal receiving module 45, a pulse signal de-modulation module 48, and a pulse signal filtering and amplifying module 46.

The pulse signal generator 41 is configured to generate the first radiated pulse signals 22 and the second radiated pulse signals 24, according to a generating instruction of a first radiated pulse signal and a second radiated pulse signal from the signal processing device 21 via the first wireless module 37, to the first antenna 13 and the second antenna 15, respectively. The first radiated pulse signals 22 and the second radiated pulse signals 24 may be modulated by the modulation module 42, and then, be sent to the transmitting module 44.

The first scattered pulse signals 26 and the second scattered pulse signals 28, from the pulse signal receiving module 45, may be de-modulated by the de-modulation module 48, and be filtered and amplified by the pulse signal filtering and amplifying module 46, before being sent to the signal processing device 21 via the first wireless module 37, respectively.

Moreover, after the first scattered pulse signals 26 and the second scattered pulse signals 28 are transmitted to the signal processing device 21, the calculation unit 311 may have an algorithm work on a plurality of calculations in order to generate a systolic blood pressure and a diastolic blood pressure of the artery.

There is a first time difference of each of the first radiated pulse signals 22 and each of the first scattered pulse signals 26. The time difference may be obtained by the following formula.

“first time difference=receiving time of the first scattered pulse signal−emitting time of the first radiated pulse signal”

There is a second time difference of each of the second radiated pulse signals 24 and each of the second scattered pulse signals 28. The time difference may be obtained by the following formula.

“second time difference=receiving time of the second scattered pulse signal−emitting time of the second radiated pulse signal”

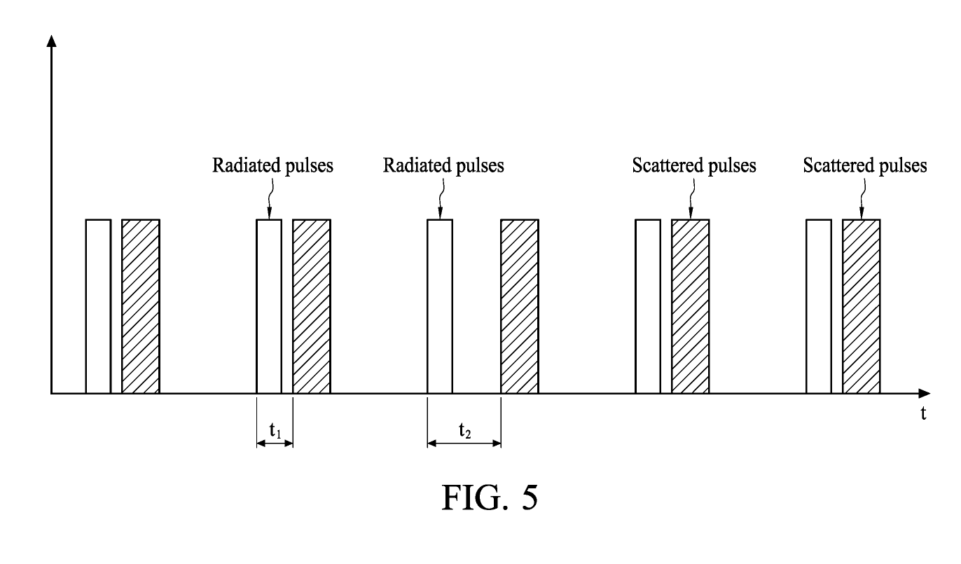

FIG. 5 shows a schematic view of time differences of a radiated pulse signal and a scattered pulse signal. As shown in FIG. 5, while a time difference t₂ is larger than a time difference t₁, a pulse peak would occur, wherein the time difference t₁ is a previous time difference to the time difference t₂.

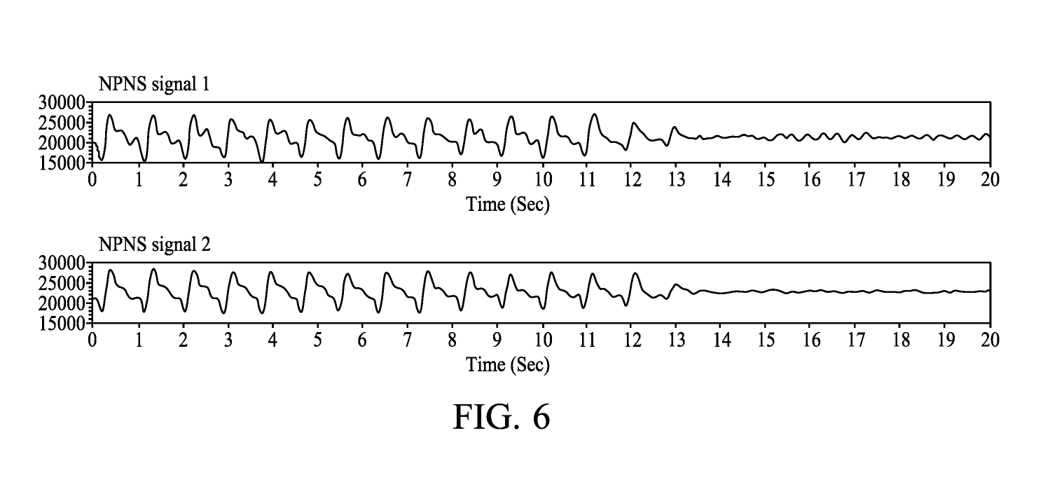

FIG. 6 shows a schematic wave form of first pulse peaks and second pulse peaks of one embodiment of the current disclosure. As shown in FIG. 6, there is a pulse time difference of the first pulse peak and the second pulse peak, which may be obtained by the following formula.

“pulse time difference=generating time of the second pulse peak−generating time of the first pulse peak”

Therefore, a pulse wave velocity (PWV) may be obtained by the following formula.

“pulse wave velocity (PWV)=a distance of the first measure point and the second measure point D/the pulse time difference”
In this embodiment, for example the distance D is in a range of 1 to 10 cm.

Moreover, the systolic blood pressure BP_{Sys} and the diastolic blood pressure BP_{Dia} of the artery may be obtained by the following formula.

BP_{Sys}=a₁+PWV+b₁

BP_{Dia}=a₂×PWV+b₂;

The a₁ and the a₂ are weighting coefficients to the PWV, and the b₁ and the b₂ are linear weighting coefficients.

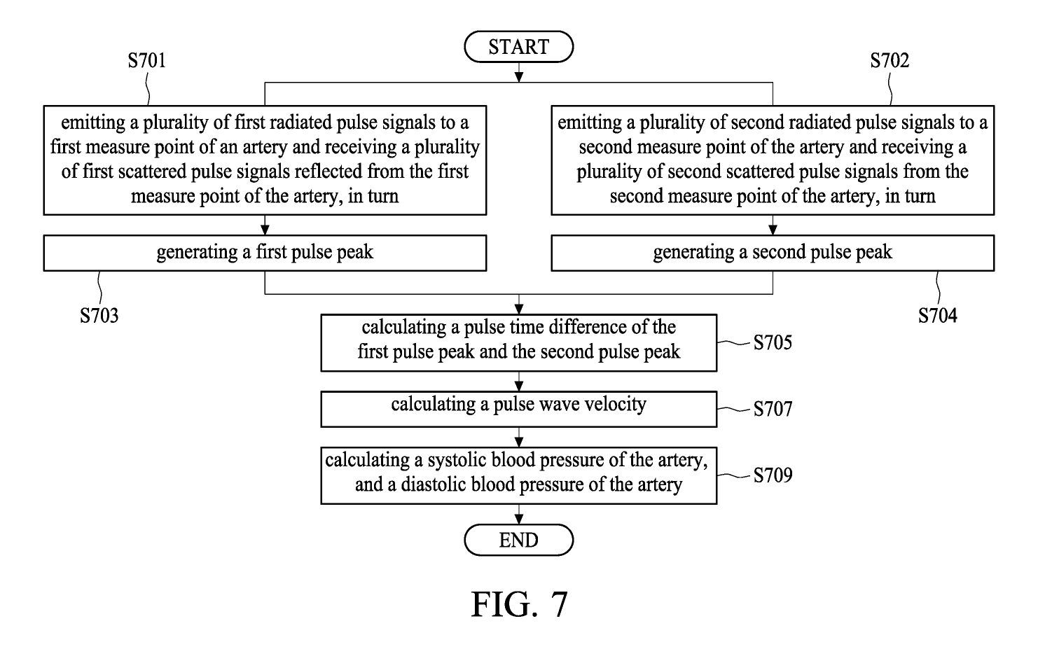

Therefore, FIG. 7 shows a flow chart of a method of a physiology measuring system of one embodiment of the current disclosure. As shown in FIG. 7, in step S701, a plurality of first radiated pulse signals may be emitted by a first antenna to a first measure point of an artery, and a plurality of first scattered pulse signals reflected from the first measure point of the artery may be received by the first antenna, in turn.

In step S702, a plurality of second radiated pulse signals may be emitted by a second antenna to a second measure point of the artery, and a plurality of second scattered pulse signals from the second measure point of the artery may be received by the second antenna, in turn. In step S703, a first pulse peak may be generated, and in step S704, a second pulse peak may be generated. In step S705, a pulse time difference may be obtained by calculating a formula “pulse time difference=generating time of the second pulse peak−generating time of the first pulse peak”.

In step S707, a pulse wave velocity (PWV) may be obtained by calculating a formula “pulse wave velocity (PWV)=a distance of the first measure point and the second measure point/the pulse time difference”, wherein the distance is in a range of 1 to 10 cm, and in step S709, a systolic blood pressure BP_{Sys} and a diastolic blood pressure BP_{Dia} of the artery may be obtained by calculating the following formula.

BP_{Sys}=a₁×PWV+b₁

BP_{Dia}=a₂×PWV+b₂;

The a₁ and the a₂ are weighting coefficients to the PWV, and the b₁ and the b₂ are linear weighting coefficients.

In step S701, a plurality of first time differences may be obtained by calculating the following formula “first time difference=receiving time of the first scattered pulse signal−emitting time of the first radiated pulse signal”. In step S702, a plurality of second time differences may be obtained by calculating the following formula “second time difference=receiving time of the second scattered pulse signal−emitting time of the second radiated pulse signal”.

In step S703, the first pulse peak is generated according to an occurrence of, a first time difference being larger than a previous first time difference, and in step S704, the second pulse peak is generated according to an occurrence of, a second time difference being larger than a previous second time difference.

Although the current disclosure and its objectives have been described in detail, it should be understood that various changes, substitutions and alterations can be made herein without departing from the spirit and scope of the disclosure as defined by the appended claims. For example, many of the processes discussed above can be implemented using different methodologies, replaced by other processes, or a combination thereof.

Moreover, the scope of the present application is not intended to be limited to the particular embodiments of the process, machine, manufacture, composition of matter, means, methods and steps described in the specification. As one of ordinary skill in the art will readily appreciate from the disclosure of the current disclosure, processes, machines, manufacture, compositions of matter, means, methods, or steps, presently existing or later to be developed, that perform substantially the same function or achieve substantially the same result as the corresponding embodiments described herein may be utilized according to the current disclosure. As such, the appended claims are intended to include within their scope such processes, machines, manufacture, compositions of matter, means, methods, or steps.

What is claimed is:

1. A method processed by a signal processing device of a physiology measuring system, comprising:
calculating a pulse time difference of a first pulse peak and a second pulse peak;
calculating a pulse wave velocity; and
calculating a systolic blood pressure of an artery, and a diastolic blood pressure of the artery;
wherein the first pulse peak and the second pulse peak is generated by a sensing device;
wherein the sensing device emits a plurality of first radiated pulse signals to a first measure point of the artery and, in turn, receiving a plurality of first scattered pulse signals reflected from the first measure point of the artery; and
wherein the sensing device emits a plurality of second radiated pulse signals to a second measure point of the artery and, in turn, receiving a plurality of second scattered pulse signals from the second measure point of the artery.

2. The method of a physiology measuring system of claim 1, wherein the step of emitting the plurality of first radiated pulse signals to the first measure point of the artery and, in turn, receiving the plurality of first scattered pulse signals reflected from the first measure point of the artery further comprises obtaining a plurality of first time differences.

3. The method of a physiology measuring system of claim 1, wherein the step of emitting the plurality of second radiated pulse signals to the second measure point of the artery and, in turn, receiving the plurality of second scattered pulse signals from the second measure point of the artery further comprises obtaining a plurality of second time differences.

4. The method of a physiology measuring system of claim 2, wherein each of the first time differences is obtained by the following formula:
first time difference=receiving time of the first scattered pulse signal−emitting time of the first radiated pulse signal.

5. The method of a physiology measuring system of claim 3, wherein each of the second time differences is obtained by the following formula:
second time difference=receiving time of the second scattered pulse signal−emitting time of the second radiated pulse signal.

6. The method of a physiology measuring system of claim 1, wherein the first pulse peak is generated according to an occurrence of, a first time difference being larger than a previous first time difference.

7. The method of a physiology measuring system of claim 1, wherein the second pulse peak is generated according to an occurrence of, a second time difference being larger than a previous second time difference.

8. The method of a physiology measuring system of claim 1, wherein the step of calculating the pulse time difference of the first pulse peak and the second pulse peak is calculated by the following formula:
pulse time difference=generating time of the second pulse peak−generating time of the first pulse peak.

9. The method of a physiology measuring system of claim 1, wherein the step of calculating the pulse wave velocity is achieved by the following formula:
pulse wave velocity (PWV)=a distance of the first measure point and the second measure point/the pulse time difference

10. The method of a physiology measuring system of claim 1, wherein the step of calculating the systolic blood pressure of the artery, and the diastolic blood pressure of the artery is achieved by the following formulas:
BP_{Sys}=a₁×PWV+b₁; and
BP_{Dia}=a₂×PWV+b₂;
wherein the pulse wave velocity (PWV) is a measure of the first measure point and the second measure point of the artery, the BP_{Sys} is the systolic blood pressure of the artery, the BP_{Dia} is the diastolic blood pressure of the artery; and
wherein a₁ and a₂ are weighting coefficients to the PWV, and b₁ and b₂ are linear weighting coefficients.

11. The method a of a physiology measuring system of claim 9, wherein the distance is in a range of 1 to 10 cm.
